(19) United States
(12) Patent Application Publication
(10) Pub. No.: US 2014/0228312 A1
(43) Pub. Date: Aug. 14, 2014

(54) ANTIBIOTIC COMPOSITION AND ITS USES

(71) Applicant: Agency for Science, Technology and Research, Singapore (SG)

(72) Inventors: Lianhui Zhang, Singapore (SG); Yinyue Deng, Singapore (SG)

(21) Appl. No.: 14/348,971

(22) PCT Filed: Oct. 1, 2012
(86) PCT No.: PCT/SG2012/000367

§ 371 Date: Apr. 1, 2014

(30) Foreign Application Priority Data
Oct. 3, 2011 (SG) 201107196-6

(51) Int. Cl. A61K 31/201; A61K 31/43; A61K 31/496; A61K 31/7036; A61K 31/20

(52) U.S. Cl. CPC A61K 31/201; A61K 31/7036; A61K 31/20; A61K 31/496; A61K 31/43
USPC 514/41; 514/40; 514/254.11; 514/192

(57) ABSTRACT

The present invention relates to a composition comprising: an antibiotic selected from the group consisting of an aminoglycoside antibiotic, a beta-lactam antibiotic, an ansamycin antibiotic, a macrolide antibiotic, a sulfonamide antibiotic, a quinolone antibiotic, an oxazolidinone antibiotic, a glycopeptide antibiotic, and a mixture thereof; and a fatty acid represented by formula (I), a stereoisomer, a salt or an ester thereof, wherein R1 is a substituted or unsubstituted aliphatic group.

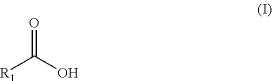

### CROSS-REFERENCE TO RELATED APPLICATION

This application claims the benefit of priority of Singapore Patent Application No. 201107196-6, filed 3 Oct. 2011, the contents of which being hereby incorporated by reference in its entirety for all purposes.

### TECHNICAL FIELD

The invention relates to antibiotic compositions, and in particular, to antibiotic compositions including a combination of antibiotic and non-antibiotic substances, the combination providing a synergistic antibacterial effect thereto.

### BACKGROUND

Antibiotics, which act by either killing or stopping microbial growth, have been used extensively in the control and prevention of infectious diseases. However, this live-or-die selection pressure has inevitably fostered the emergence of superbugs that are resistant to multidrugs. Infections associated with antibiotic-resistant pathogens are becoming more and more common in clinical and nosocomial settings (Pfaller et al., 1998; Livermore, 2004), which has become a great healthy care and public concerns.

In addition, antibiotics are associated with a range of adverse effects (Slama et al., 2005). These side-effects are varied and dependent on the antibiotics used, the dosage, the microbial organisms targeted, and the individual patient. For instance, treatment using aminoglycoside antibiotics, such as gentamicin and kanamycin, can cause serious symptoms, including balance difficulty, hearing loss, and nephrotoxicity (Sundin et al., 2001; Giannini et al., 1978). Reduction and limitation of antibiotic usage is therefore of critical importance in clinical treatment of microbial infections.

Resistance to antibiotics is the functional connections at multiple levels, such as at the level of gene expression, genetic interactions and protein interactions (Parsons et al., 2004; Costanzo et al., 2010). Combination antibiotics containing more than one antibiotic agent are designed to either improve efficacy through synergistic action of the agents, or to overcome the bacterial resistance. This method has been effectively used for the treatments of tuberculosis, leprosy, malaria, HIV, infections associated with cystic fibrosis, and infective endocarditis (Aaron et al., 2000; Le and Bayer, 2003; Athamna, et al., 2005; Ejim et al., 2011).

Currently, antibiotic combinations are frequently used to provide empirical treatment for serious infections. However, discovery of effective antibiotic combinations is still limited in scope. Given the rapid emergence of resistant bacteria and changing landscape of antibiotic resistance, it is essential to continue searching for effective antibiotic combinations and other novel approaches to control infectious diseases.

### SUMMARY

According to a first aspect, there is provided a composition, comprising:
an antibiotic selected from the group consisting of an aminoglycoside antibiotic, a beta-lactam antibiotic, an ansamycin antibiotic, a macrolide antibiotic, a sulfonamide antibiotic, a quinolone antibiotic, an oxazolidinone antibiotic, a glycopeptide antibiotic, and a mixture thereof; and
a fatty acid represented by formula (I), a stereoisomer, a salt or an ester thereof,

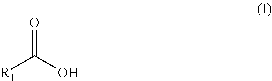

wherein R₁ is a substituted or unsubstituted aliphatic group.

A second aspect relates to a pharmaceutical composition comprising the composition of the first aspect and a pharmaceutically acceptable carrier.

In a third aspect, there is disclosed use of the composition of the first aspect or the pharmaceutical composition of the second aspect for the manufacture of a medicament for treating or preventing a bacterial infection.

A fourth aspect provides a method of treating or preventing a bacterial infection in a subject, comprising administering a therapeutically effective amount of the composition of the first aspect or the pharmaceutical composition of the second aspect to a subject in need thereof.

In a fifth aspect, there is disclosed a method of supporting antibiotic therapy or prophylaxis in a subject, comprising administering a therapeutically effective amount of a fatty acid represented by formula (I), a stereoisomer, a salt or an ester thereof,

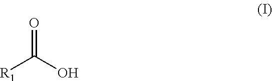

wherein R₁ is a substituted or unsubstituted aliphatic group, and

at least one antibiotic to a subject in need thereof, wherein the antibiotic is selected from the group consisting of an aminoglycoside antibiotic, a beta-lactam antibiotic, an ansamycin antibiotic, a macrolide antibiotic, a sulfonamide antibiotic, a quinolone antibiotic, an oxazolidinone antibiotic, a glycopeptide antibiotic, and a mixture thereof.

### BRIEF DESCRIPTION OF THE DRAWINGS

In the drawings, like reference characters generally refer to the same parts throughout the different views. The drawings are not necessarily drawn to scale, emphasis instead generally being placed upon illustrating the principles of various embodiments. In the following description, various embodiments of the invention are described with reference to the following drawings.

FIG. 1 shows the influences of exogenous addition of 50 μM DSF-family signals on the growth rate of (A) B. cereus, (B) B. thuringiensis, (C) N. subflava, (D) S. aureus, and (E) M. smegmatis. The error bars show the standard deviations of three repeats.

FIG. 2 shows that exogenous addition of 50 μM DSF reduces the minimum inhibitory concentrations (MICs) of (A) B. thuringiensis, (B) N. subflava, (C) S. aureus, and (D) M. smegmatis. The data shown are the means of two repeats, and error bars indicate standard deviations.

FIG. 3 shows that the addition of DSF enhances the susceptibility of P. aeruginosa to gentamicin and kanamycin: (A) Growth curves of PAO1 with addition of methanol (▪) and DSF (▴), respectively; (B) Growth curves of PAO1 with addition of gentamicin at the final concentration of 2 nM in the presence (▴) and absence (▪) of 50 μM DSF; (C) Growth curves of PAO1 with addition of kanamycin at the final concentration of 0.2 μM in the presence (▴) and absence (▪) of 50 μM DSF. The error bars show the standard deviations of three repeats.

### DESCRIPTION

The following detailed description refers to the accompanying drawings that show, by way of illustration, specific details and embodiments in which the invention may be practised. These embodiments are described in sufficient detail to enable those skilled in the art to practise the invention. Other embodiments may be utilized and structural and other changes may be made without departing from the scope of the invention. The various embodiments are not necessarily mutually exclusive, as some embodiments can be combined with one or more other embodiments to form new embodiments.

Using non-antibiotic molecules to enhance the antibacterial efficacy of antibiotics offers a new kind of opportunity to practise a previously untapped expanse of clinical treatments. A few combinations of non-antibiotics with antibiotics showed increased activity against bacterial pathogens in vitro and in vivo (Kristiansen et al., 2007; Lehtinen and Lilius, 2007; Mazumdar et al., 2009; Ejim et al., 2011).

The present invention relates to the identification of a class of fatty acid compounds as novel adjuvants to conventional antibiotics for the therapy of infectious diseases. The combination of the antibiotic and non-antibiotic compounds produces a synergistic antibacterial effect wherein the non-antibiotic fatty acid compounds enhance the antibacterial efficacy of the antibiotic.

Thus, according to a first aspect, there is provided a composition comprising an antibiotic and a fatty acid represented by formula (I), a stereoisomer, a salt or an ester thereof,

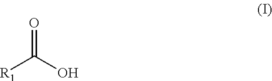

wherein R₁ is a substituted or unsubstituted aliphatic group. The antibiotic is selected from the group consisting of an aminoglycoside antibiotic, a beta-lactam antibiotic, an ansamycin antibiotic, a macrolide antibiotic, a sulfonamide antibiotic, a quinolone antibiotic, an oxazolidinone antibiotic, a glycopeptide antibiotic, and a mixture thereof.

The term “synergistic”, as used hereinafter, is meant an antibacterial effect created from the application of a combination of antibiotic and non-antibiotic (fatty acid) substances to produce an antibacterial effect that is greater than the sum of the antibacterial effects produced by the application of the individual substance. In particular, the present non-antibiotic fatty acid compounds enhance the antibacterial efficacy of the antibiotic.

In various embodiments, the fatty acids are free fatty acids, meaning that the fatty acid molecules are not bound to another molecule.

In alternative embodiments, the fatty acids are not free and are bound to another molecule. For example, the fatty acid may be associated with or conjugated to another molecule, such as a peptide. In certain embodiments, the molecule bound to the fatty acid does not adversely affect the synergistic effect produced by the combination of antibiotic and the fatty acid. In other words, the bound molecule does not adversely affect the enhancement of the antibacterial effect of the antibiotic by the fatty acid.

In the present context, the term “aliphatic”, alone or in combination, refers to a straight chain or branched chain hydrocarbon comprising at least one carbon atom, and may be saturated or mono- or poly-unsaturated and can include heteroatoms. A saturated aliphatic group has no double or triple bonds. An unsaturated aliphatic group contains one or more double and/or triple bonds (alkenyl or alkynyl moieties). The branches of the hydrocarbon chain may include linear chains as well as non-aromatic cyclic elements. The hydrocarbon chain, which may, unless otherwise stated, be of any length, and contain any number of branches. Typically, the hydrocarbon (main) chain includes 1 to 5, to 10, to 15 or to 20 carbon atoms. Both the main chain as well as the branches may furthermore contain heteroatoms as for instance N, O, S, Se or Si or carbon atoms may be replaced by these heteroatoms. The aliphatic group used herein is meant to include both substituted and unsubstituted forms of the respective moiety. Substituents may be any functional group, as for example, but not limited to, methyl, amino, amido, azido, carbonyl, carboxyl, cyano, isocyano, dithiane, halogen, hydroxyl, nitro, organometal, organoboron, seleno, silyl, silano, sulfonyl, thio, thiocyano, trifluoromethyl sulfonyl, p-toluenesulfonyl, bromobenzenesulfonyl, nitrobenzenesulfonyl, and methane-sulfonyl.

The term “alkyl”, alone or in combination, refers to a saturated aliphatic hydrocarbon including straight chain, or branched chain groups. Preferably, the alkyl group has 1 to 20 carbon atoms (whenever a numerical range; e.g. “1-20” or “C₁-C₂₀” is stated herein wherein the numerical range is stated in the subscript, it means that the group, in this case the alkyl group, may contain 1, 2, 3, 4, 5, 6, 7, 8, 9, 10, 11, 12, 13, 14, 15, 16, 17, 18, 19, or 20 carbon atoms). Examples of alkyl groups include, but are not limited to, methyl, ethyl, propyl, n-propyl, isopropyl, butyl, n-butyl, isobutyl, sec-butyl, tert-butyl, tert-amyl, pentyl, hexyl, heptyl, octyl, nonyl, decyl, undecyl, dodecyl, tridecyl, tetradecyl, pentadecyl, and the like. More specifically, the alkyl group may be a higher alkyl having 5 to 20 carbon atoms. Preferably, the alkyl group may have a medium size alkyl having 7 to 14 carbon atoms. The alkyl group may be substituted or unsubstituted. When substituted, the substituent group(s) is one or more, for example, one, two, three, four, or five groups, individually selected from the group consisting of alkyl, heteroalkyl, haloalkyl, heteroholoalkyl, cycloalkyl, aryl, arylalkyl, heteroaryl, non-aromatic heterocycle, hydroxy, alkoxy, aryloxy, mercapto, alkylthio, arylthio, cyano, halo, carbonyl, thiocarbonyl, O-carbamyl, N-carbamyl, O-thiocarbamyl, N-thiocarbamyl, C-amido, N-amido, S-sulfonamido, N-sulfonamido, C-carboxy, O-carboxy, isocyanato, thiocyanato, isothiocyanato, nitro, silyl, trihalomethanesulfonyl, and amino, including mono- and di-substituted amino groups. In embodiments in which two or more hydrogen atoms have been substituted, the substituent groups may be linked to form a ring.

The term “alkenyl” as used herein refers to an alkyl group that contains in the straight or branched hydrocarbon chain one or more double bonds. In certain embodiments, an alkenyl comprises 2 to 20 carbon atoms, for example 5 to 20 carbon atoms or 7 to 14 carbon atoms, wherein a numerical range, such as “5 to 20” or “C₅-C₂₀” wherein the numerical range is stated in the subscript, refers to each integer in the given range, e.g. “C₅-C₂₀ alkenyl” means that an alkenyl group comprising 5, 6, 7, 8, 9, 10, 11, 12, 13, 14, 15, 16, 17, 18, 19, or 20 carbon atoms. An alkenyl used in this invention can for example be substituted or unsubstituted. When substituted, the substituent group(s) can be as defined above. Examples of alkenyl groups include, but are not limited to, ethenyl, propenyl, butenyl, 1,4-butadienyl, pentenyl, 4-methylhex-1-enyl, 4-ethyl-2-methylhex-1-enyl and the like.

The term “alkynyl” as used herein refers to an alkyl group that contains in the straight or branched hydrocarbon chain one or more triple bonds. In certain embodiments, an alkynyl comprises 2 to 6 carbon atoms, for example 2 to 6 carbon atoms, 2 to 5 carbon atoms, or 2 to 4 carbon atoms, wherein a numerical range, such as “2 to 6” or “C₂-C₆” wherein the numerical range is stated in the subscript, refers to each integer in the given range, e.g. “C₂-C₆ alkynyl” means that an alkynyl group comprising 2 carbon atoms, 3 carbon atoms, etc., up to and including 6 carbon atoms. An alkynyl group of this invention may be optionally substituted. Examples of alkynyl groups include, but are not limited to, ethynyl, propynyl, butynyl, and the like. An alkenyl group used in this invention can for example be substituted or unsubstituted. When substituted, the substituent group(s) can be as defined above.

The term “cycloalkyl” refers to a completely saturated hydrocarbon ring. For example, the cycloalkyl group may range from C₃ to C₈. Examples of cycloalkyl groups include, but are not limited to, cyclopropyl, cyclobutyl, cyclopentyl, cyclohexyl, and the like. An cycloalkyl group can for example be substituted or unsubstituted. When substituted, the substituent group(s) can be as defined above.

The term “alkoxy”, alone or in combination, refers to an aliphatic hydrocarbon having an alkyl-O-moiety. Examples of alkoxy groups include, but are not limited to, methoxy, ethoxy, propoxy, butoxy, and the like. An alkoxy group can for example be substituted or unsubstituted. When substituted, the substituent group(s) can be as defined above.

A “cycloalkoxy” group refers to an —O-cycloalkyl group, as defined herein. One example is cyclopropyloxy. A cycloalkoxy group used in this invention can for example be substituted or unsubstituted. When substituted, the substituent group(s) can be as defined above.

An “aryl” group refers to an all-carbon monocyclic or fused-ring polycyclic (i.e. rings which share adjacent pairs of carbon atoms) groups of 6 to 14 ring atoms and having a completely conjugated pi-electron system. Examples, without limitation, of aryl groups are phenyl, naphthalenyl and anthracenyl. The aryl group may be substituted or unsubstituted. When substituted, the substituted group(s) is one or more, for example one, two, or three substituents, independently selected from the group consisting of C₁-C₁₀ alkyl, C₃-C₈ cycloalkyl, C₆-C₁₄ aryl, 5-10 membered heteroaryl wherein 1 to 4 ring atoms are independently selected from nitrogen, oxygen or sulfur, 5-10 membered heteroalicyclic wherein 1 to 3 ring atoms are independently nitrogen, oxygen or sulfur, hydroxy, alkoxy, C₃-C₈ cycloalkoxy, aryloxy, mercapto, alkylthio, arylthio, cyano, halo, trihalomethyl, carbonyl, thiocarbonyl, O-carbamyl, N-carbamyl, O-thiocarbamyl, N-thiocarbamyl, C-amido, N-amido, C-carboxy, O-carboxy, nitro, silyl, sulfinyl, sulfonyl, or amino. Preferably the substituent(s) is/are independently selected from chloro, fluoro, bromo, methyl, ethyl, hydroxy, methoxy, nitro, carboxy, methoxycarbonyl, sulfonyl, or amino.

A “heteroaryl” group refers to a monocyclic or fused aromatic ring (i.e., rings which share an adjacent pair of atoms) of 5 to 10 ring atoms in which one, two, three or four ring atoms are selected from the group consisting of nitrogen, oxygen and sulfur and the rest being carbon. Examples, without limitation, of heteroaryl groups are pyridyl, pyrrolyl, furyl, thienyl, imidazolyl, oxazolyl, isoxazolyl, thiazolyl, isothiazolyl, pyrazolyl, 1,2,3-triazolyl, 1,2,4-triazolyl, 1,2,3-oxadiazolyl, 1,2,4-oxadiazolyl, 1,2,5-oxadiazolyl, 1,3,4-oxadiazolyl, 1,3,4-triazinyl, 1,2,3-triazinyl, benzofuryl, isobenzofuryl, benzothienyl, benzotriazolyl, isobenzothienyl, indolyl, isoindolyl, 3H-indolyl, benzimidazolyl, benzothiazolyl, benzoxazolyl, quinolizinyl, quinazolinyl, pthalazinyl, quinoxalinyl, cinnnolinyl, napthyridinyl, quinolyl, isoquinolyl, tetrazolyl, 5,6,7,8-tetrahydroquinolyl, 5,6,7,8-tetra-hydroisoquinolyl, purinyl, pteridinyl, pyridinyl, pyrimidinyl, carbazolyl, xanthenyl or benzoquinolyl. The heteroaryl group may be substituted or unsubstituted. When substituted, the substituted group(s) is one or more, for example one or two substituents, independently selected from the group consisting of C₁-C₁₀ alkyl, C₃-C₈ cycloalkyl, C₆-C₁₄ aryl, 5-10 membered heteroaryl wherein 1 to 4 ring atoms are independently selected from nitrogen, oxygen or sulfur, 5-10 membered heteroalicyclic wherein 1 to 3 ring atoms are independently nitrogen, oxygen or sulfur, hydroxy, C₁-C₁₀ alkoxy, C₃-C₈ cycloalkoxy, aryloxy, mercapto, alkylthio, arylthio, cyano, halo, trihalomethyl, carbonyl, thiocarbonyl, O-carbamyl, N-carbamyl, O-thiocarbamyl, N-thiocarbamyl, C-amido, N-amido, C-carboxy, O-carboxy, nitro, silyl, sulfinyl, sulfonyl, or amino. Preferably the substituent(s) is/are independently selected from chloro, fluoro, bromo, methyl, ethyl, hydroxy, methoxy, nitro, carboxy, methoxycarbonyl, sulfonyl, or amino.

A “heteroalicyclic” group refers to a monocyclic or fused ring of 5 to 10 ring atoms containing one, two, or three heteroatoms in the ring which are selected from the group consisting of nitrogen, oxygen and —S(O)ₙ where n is 0-2, the remaining ring atoms being carbon. The rings may also have one or more double bonds. However, the rings do not have a completely conjugated pi-electron system. Examples, without limitation, of heteroalicyclic groups are pyrrolidine, piperidine, piperazine, morpholine, imidazolidine, tetrahydropyridazine, tetrahydrofuran, thiomorpholine, tetrahydropyridine, and the like. The heteroalicyclic ring may be substituted or unsubstituted. When substituted, the substituted group (s) is one or more, for example one, two, or three substituents, independently selected from the group consisting of C₁-C₁₀ alkyl, C₃-C₈ cycloalkyl, C₆-C₁₄ aryl, 5-10 membered heteroaryl wherein 1 to 4 ring atoms are independently selected from nitrogen, oxygen or sulfur, 5-10 membered heteroalicyclic wherein 1 to 3 ring atoms are independently nitrogen, oxygen or sulfur, hydroxy, C₁-C₁₀ alkoxy, C₃-C₈ cycloalkoxy, aryloxy, mercapto, alkylthio, arylthio, cyano, halo, trihalomethyl, carbonyl, thiocarbonyl, O-carbamyl, N-carbamyl, O-thiocarbamyl, N-thiocarbamyl, C-amido, N-amido, C-carboxy, O-carboxy, nitro, silyl, sulfinyl, sulfonyl, or amino. The substituent(s) is/are for example independently selected from chloro, fluoro, bromo, methyl, ethyl, hydroxy, methoxy, nitro, carboxy, methoxycarbonyl, sulfonyl, or amino.

The term “heteroatom” refers to an atom other than carbon or hydrogen. Heteroatoms are typically independently selected from oxygen, sulphur, nitrogen, and phosphorus, but are not limited to those atoms. In embodiments in which two or more heteroatoms are present, the two or more heteroatoms may all be the same as one another, or some or all of the two or more heteroatoms may each be different from the others.

An “aryloxy” group refers to both an —O-aryl and an —O-heteroaryl group, as defined herein. Examples include and are not limited to phenoxy, napthyloxy, pyridyloxy, furanyloxy, and the like.

A “mercapto” group refers to an —SH group.

An “alkylthio” group refers to both an S-alkyl and an —S-cycloalkyl group, as defined herein. Examples include and are not limited to methylthio, ethylthio, and the like.

An “arylthio” group refers to both an —S-aryl and an —S-heteroaryl group, as defined herein. Examples include and are not limited to phenylthio, napthylthio, pyridylthio, furanylthio, and the like.

A “cyano” group refers to a —CN group.

A “halo” or “halogen” group refers to fluorine, chlorine, bromine or iodine.

A “trihalomethyl” group refers to a —CX₃ group wherein X is a halo group as defined herein e.g., trifluoromethyl, trichloromethyl, tribromomethyl, dichlorofluoromethyl, and the like.

“Carbonyl” refers to a —C(═O)—R″ group, where R″ is selected from the group consisting of hydrogen, alkyl, cycloalkyl, aryl, heteroaryl (bonded through a ring carbon) and heteroalicyclic (bonded through a ring carbon), as defined herein. Representative examples include and the not limited to acetyl, propionyl, benzoyl, formyl, cyclopropylcarbonyl, pyridinylcarbonyl, pyrrolidin-1 ylcarbonyl, and the like.

A “thiocarbonyl” group refers to a —C(═S)—R″ group, with R″ as defined herein.

An “O-carbamyl” group refers to a —OC(═O)NR″R′″ group, with R″ as defined herein, R′″ having the same definition as R″ and may be the same or different from R″.

An “N-carbamyl” group refers to a R″OC(═O) NR′″-group, with R″ and R′″ as defined above.

An “O-thiocarbamyl” group refers to a —OC(═S)NR″R′″ group, with R″ and R′″ as defined above.

An “N-thiocarbamyl” group refers to a R″OC(═S)NR′″-group, with R″ and R′″ as defined above.

A “C-amido” group refers to a —C(═O)NR″R′″ group, with R″ and R′″ as defined herein. For example, R″ is hydrogen or unsubstituted C₁-C₄ alkyl and R′″ is hydrogen, C₁-C₄ alkyl optionally substituted with heteroalicyclic, hydroxy, or amino. For example, C(═O)NR″R′″ may be aminocarbonyl, dimethylaminocarbonyl, diethylaminocarbonyl, diethylaminoethylaminocarbonyl, ethylaminoethylaminocarbonyl, and the like.

A “nitro” group refers to a —NO₂ group.

A “sulfinyl” group refers to a —S(O)—R″ group, wherein R″ is as defined herein.

A “sulfonyl” group refers to a —S(O)₂R″ group wherein, R″ is as defined herein.

The compounds of and used in the invention are inclusive of all possible stereo-isomers of the respective compounds, including tautomers, geometric isomers, e.g. Z and E isomers (cis and trans isomers), and optical isomers, e.g. diastereomers and enantiomers. Furthermore, the invention includes in its scope both the individual isomers and any mixtures thereof, e.g. racemic mixtures. In this context, the term “isomer” is meant to encompass all optical isomers of the compounds described herein. It will be appreciated by those skilled in the art that the compounds described herein may contain at least one chiral center. Accordingly, the compounds of the invention may exist in optically active or racemic forms. It is to be understood that the compounds according to the present invention may encompass any racemic or optically active form, or mixtures thereof. In one embodiment, the compounds of the invention can be pure (R)-isomers. In another embodiment, the compounds of the invention can be pure (S)-isomers. In another embodiment, the compounds of the invention can be a mixture of the (R) and the (S) isomers. In a further embodiment, the compounds of the invention can be a racemic mixture comprising an equal amount of the (R) and the (S) isomers. The individual isomers may be obtained using the corresponding isomeric forms of the starting material or they may be separated after the preparation of the end compound according to conventional separation methods. For the separation of optical isomers, e.g. enantiomers, from the mixture thereof, the conventional resolution methods for example fractional crystallization may be used.

As used herein, the term “salt” (or otherwise termed as “pharmaceutically acceptable salt”) in connection with the fatty acid represented by formula (I), refers to those salts which retain the biological effectiveness and properties of the parent compound of formula (I). Such salts include, but are not restricted to: (1) an acid addition salt which is obtained by reaction of the free base of the parent compound with inorganic acids such as hydrochloric acid, hydrobromic acid, nitric acid, phosphoric acid, sulfuric acid, and perchloric acid and the like, or with organic acids such as acetic acid, oxalic acid, (D) or (L) malic acid, maleic acid, methanesulfonic acid, ethanesulfonic acid, p-toluenesulfonic acid, salicylic acid, tartaric acid, citric acid, succinic acid or malonic acid and the like, preferably hydrochloric acid or (L)-malic acid; or (2) salts formed when an acidic proton present in the parent compound either is replaced by a metal ion, e. g., an alkali metal ion, such as sodium or potassium, an alkaline earth ion, such as magnesium or calcium, or an aluminum ion; or coordinates with an organic base such as ethanolamine, diethanolamine, triethanolamine, tromethamine, N-methylglucamine, and the like.

The term “ester” refers to a chemical moiety with formula —(R)n-COOR′, where R and R′ are independently selected from alkyl, cycloalkyl, aryl, heteroaryl (bonded through a ring carbon) and non-aromatic heterocycle (bonded through a ring carbon), where n is 0 or 1. For example, the carboxylic acid group of the fatty acid may be esterified by an alcohol, such as methanl, ethanol, or propanol, to form the ester. In certain embodiments, it may be advantageous to provide the fatty acid in a form of an ester for easy handling, for example, whereby the ester may be hydrolysed in vivo to form the fatty acid.

The aliphatic group of R₁ may be a straight or branched hydrocarbon chain, as defined above. The aliphatic group of R₁ may also be substituted or unsubstituted. For example, the aliphatic group is substituted with at least one substituent selected from the group consisting of hydroxy, heteroalkyl, haloalkyl, heterohaloalkyl, cycloalkyl, aryl, arylalkyl, heteroaryl, non-aromatic heterocycle, alkoxy, aryloxy, mercapto, alkylthio, arylthio, cyano, halo, carbonyl, thiocarbonyl, O-carbamyl, N-carbamyl, O-thiocarbamyl, N-thiocarbamyl, C-amido, N-amido, S-sulfonamido, N-sulfonamido, C-carboxy, O-carboxy, isocyanato, thiocyanato, isothiocyanato, nitro, silyl, trihalomethanesulfonyl, amino, and a combination thereof.

In certain embodiments, the aliphatic group of R₁ is a straight hydrocarbon chain. For example, the straight hydrocarbon chain may be, but is not limited to, n-propyl, isopropyl, or n-butyl.

In other embodiments, the aliphatic group of R₁ is a branched hydrocarbon chain. For example, the branched hydrocarbon chain may be isopropyl, isobutyl, sec-butyl, tert-butyl, tert-amyl, or the like.

The aliphatic group of R₁ may be saturated or unsaturated.

In various embodiments, the aliphatic group of R₁ is saturated. The aliphatic group may also be a straight or branched hydrocarbon chain. In further embodiments, the aliphatic group of R₁ is saturated and R₁ is an alkyl group. In yet further embodiments, the aliphatic group of R₁ is an unsubstituted or substituted C₅-C₂₀ alkyl group. The substitutent of the alkyl group may be as defined above. In one exemplary embodiment, R₁ is a straight-chained, saturated, and unsubstituted C₁₁ alkyl group illustrated as Compound S12 in Table 1.

In alternative embodiments, the aliphatic group of R₁ is unsaturated. The aliphatic group may also be a straight or branched hydrocarbon chain. The unsaturated R₁ may be mono-saturated or poly-saturated. In further embodiments, the aliphatic group of R₁ is unsaturated and R₁ is an alkenyl group. In yet further embodiments, the aliphatic group of R₁ is an unsubstituted or substituted C₅-C₂₀ alkenyl group. The substitutent of the alkenyl group may be as defined above. In exemplary embodiments, R₁ is an unsubstituted straight or branched C₆, or C₇, or C₈, or C₉, or C₁₀, or C₁₁, or C₁₂, or C₁₃ alkenyl group.

In various embodiments, the C₅-C₂₀ alkenyl group comprises at least one double bond. The C₅-C₂₀ alkenyl group may comprise only one double bond. Alternatively, the C₅-C₂₀ alkenyl group may comprise two or more double bonds in the main chain. For example, the C₅-C₂₀ alkenyl group comprises at least one double bond at the carbon-2 position of the fatty acid, such as only one double bond at the carbon-2 position, or one double bond at the carbon-2 position and another double bond at the carbon-4, carbon-5, carbon-6, or carbon-7, or other position. It is to be understood and appreciated that if the fatty acid is mono-unsaturated, the double bond may likewise be located at any position other than carbon-2 position. On the same token, if the fatty acid is poly-unsaturated, the respective double bonds may be found at positions other than those mentioned above, which are given as illustrations without limiting the scope.

In various embodiments, each of the double bond of the unsaturated aliphatic group of R₁ is a cis configuration. In certain embodiments, the C₅-C₂₀ alkenyl group of R₁ is straight-chained, mono-unsaturated, and unsubstituted, wherein the double bond is located at the carbon-2 position of the fatty acid, and wherein fatty acid has a cis configuration, such as but is not limited to, Compound C8, or C10, or C11, or C12, or C13, or C14, or C15 of Table 1. In another embodiment, the C₅-C₂₀ alkenyl group of R₁ is mono-unsaturated, unsubstituted, and branched, wherein the double bond is located at the carbon-2 position of the fatty acid, and wherein fatty acid has a cis configuration, such as but is not limited to, Compound DSF of Table 1.

In alternative embodiments, each of the double bond of the unsaturated aliphatic group of R₁ is a trans configuration. In certain embodiments, the C₅-C₂₀ alkenyl group of R₁ is straight-chained, mono-unsaturated, and unsubstituted, wherein the double bond is located at the carbon-2 position of the fatty acid, and wherein fatty acid has a trans configuration, such as but is not limited to, Compound T8, or T10, or T11, or T12, or T13, or T14, or T15 of Table 1. In other embodiments, the C₅-C₂₀ alkenyl group of R₁ is branched, mono-unsaturated, and unsubstituted, wherein the double bond is located at the carbon-2 position of the fatty acid, and wherein fatty acid has a trans configuration.

In various embodiments, the fatty acid is Compound T8, or T10, or T11, or T12, or T13, or T14, or T15, or C8, or C10, or C11, or C12, or C13, or C14, or C15, or S12, or DSF of Table 1.

In various embodiments, the antibiotic is an aminoglycoside antibiotic. Examples of an aminoglycoside antibiotic include, but are not limited to, kanamycin, gentamicin, neomycin, paromomycin, amikacin, netilmycin, streptomycin, tobramycin, hydromycin, spectinomycin or combinations thereof.

In certain embodiments, the aminoglycoside antibiotic is kanamycin or gentamicin.

In embodiments where the aminoglycoside antibiotic is kanamycin, the fatty acid is selected from the group consisting of T14, C14, C15, T12, C11, C13, DSF, T11, S12, T13, T10, C10, C12, T8, T15, C8, and a combination thereof. For example, the composition may comprise kanamycin and T14 fatty acid.

In embodiments where the aminoglycoside antibiotic is gentamicin, the fatty acid is selected from the group consisting of T14, C15, C13, C14, T12, T11, C11, DSF, T13, C12, S12, T10, C10, and a combination thereof. For example, the composition may comprise gentamicin and T14 or C15 fatty acid.

In various embodiments, the antibiotic is a beta-lactam antibiotic. Examples of a beta-lactam antibiotic include, but are not limited to, penicillins, cephalosporins, carbapenems, monobactams, bridged monobactams or combinations thereof. Pencilins include but are limited to benzathine penicillin, benzylpenicillin, phenoxymethylpenicillin, procaine penicillin, oxacillin, methicillin, dicloxacillin, flucloxacillin, temocillin, amoxicillin, ampicillin, co-amoxiclav, azlocillin, carbenicillin, ricarcillin, mezlocillin, piperacillin, apalcillin, hetacillin, bacampicillin, sulbenicillin, mecicilam, pevmecillinam, ciclacillin, talapicillin, aspoxicillin, cloxacillin, nafcillin, pivampicillin or combinations thereof. Cephalosporins include, but are not limited to, cephalothin, cephaloridin, cefaclor, cefadroxil, cefamandole, cefazolin, cephalexin, cephradine, ceftizoxime, cefoxitin, cephacetril, cefotiam, cefotaxime, cefsulodin, cefoperazone, or combinations thereof. Carbapenems include, but are not limited to imipenem, meropenem, ertapenem, faropenem, doripenem, biapenem, panipenem, anti-MRSA carbapenems or combinations thereof. Monobactams include, but are not limited to, aztreonam, carumonam or the like.

In certain embodiments, the beta-lactam antibiotic is ampicillin.

In embodiments where the beta-lactam antibiotic is ampicillin, the fatty acid is selected from the group consisting of C15, C11, T11, T14, and a combination thereof. For example, the composition may comprise ampicillin and C15 fatty acid.

In various embodiments, the antibiotic is an ansamycin antibiotic. Examples of an ansamycin antibiotic include, but are not limited to streptovaricin, geldanamycin, herbimycin, rifamycin, particularly, rifampicin, rifampin, rifabutin, rifapentine or rifamixin.

In certain embodiments, the ansamycin antibiotic is rifampicin.

In embodiments where the ansamycin antibiotic is rifampicin, the fatty acid is selected from the group consisting of C11, C15, T11, C10, C12, C13, T10, T13, T15, C8, C14, DSF, S12, T12, T14, and a combination thereof. For example, the composition may comprise rifampicin and C11 or C15 fatty acid.

In various embodiments, the antibiotic is a macrolide antibiotic. Examples of a macrolide antibiotic include, but are not limited to, azithromycin, clarithromycin, dirithromycin, erythromycin, erythromycin A, erythromycin B, erythromycin C, erythromycin D, erythromycin E, erythromycin estolate, roxithromycin, troleandomycin, telithromycin, spectinomycin, methymycin, neomethymycin, erythronolid, megalomycin, picromycin, narbomycin, oleandomycin, triacetyl-oleandomycin, laukamycin, kujimycin A, albocyclin or cineromycin B.

In various embodiments, the antibiotic is a sulfonamide antibiotic. Examples of a sulfonamide antibiotic include, but are not limited to, sulfanilamide, sulfacetarnide, sulfapyridine, sulfathiazole, sulfadiazine, sulfamerazine, sulfadimidine, sulfasomidine, sulfasalazine, mafenide, sulfamethoxazole, sulfamethoxypyridazine, sulfadimethoxine, sulfasymazine, sulfadoxine, sulfametopyrazine, sulfaguanidine, succinylsulfathiazole or phthalylsulfathiazole.

In various embodiments, the antibiotic is a quinolone antibiotic. Examples of a quinolone antibiotic include, but are not limited to, oxolinic acid, cinoxacin, flumequine, miloxacin, rosoxacin, pipemidic acid, norfloxacin, enoxacin, ciprofloxacin, ofloxacin, lomefloxcain, temafloxacin, fleroxacin, pefloxacin, amifloxacin, sparfloxacin, levofloxacin, clinafloxacin or nalidixic acid.

In various embodiments, the antibiotic is an oxazolidinone antibiotic. Examples of an oxazolidinone antibiotic include, but are not limited to, linezolid or the like.

In various embodiments, the antibiotic is a glycopeptide antibiotic. Examples of a glycopeptide antibiotic include, but are not limited to vancomycin, teicoplanin, daptomycin or ramoplanin.

In addition, the present compositions may be used as antibacterial ingredients in personal hygiene articles, toiletries or cosmetics. An example of such toiletries may include oral hygiene products. An oral hygiene product refers to any composition which is used in the mouth in order to promote oral hygiene. These compositions may be in the form of aqueous solutions, for example a mouth wash composition; gels, for example toothpaste or dentrifice compositions; solids, for example lozenges; or combined with fillers, for example chewing gum compositions. In this context, a dentrice refers to a paste, liquid or powder used to help maintain acceptable oral hygiene. Exemplary personal hygiene articles include but are not limited to soaps, shampoos, shower gels, ointments, creams, lotions, deodorants and disinfectants and storage solutions for contact lenses. Examples of cosmetics include foundation make-up, eye liner, lip stick, lip gloss to mention only a few.

In another aspect, a pharmaceutical composition comprising the present composition and a pharmaceutically acceptable carrier is provided.

The pharmaceutical composition may be administered to a human patient (or subject) in which the present composition is mixed with suitable carriers or excipient(s). As used herein, “administer” or “administration” refers to the delivery of a pharmaceutical composition containing the present composition to an organism for the purpose of prevention or treatment of a bacterial infection.

Suitable routes of administration may include, without limitation, oral, rectal, transmucosal or intestinal administration or intramuscular, subcutaneous, intramedullary, intrathecal, direct intraventricular, intravenous, intravitreal, intraperitoneal, intranasal, or intraocular injections. The preferred routes of administration are oral and parenteral.

Alternatively, one may administer the compound in a local rather than systemic manner, for example, via injection of the compound directly into a vessel, optionally in a depot or sustained release formulation.

Present pharmaceutical compositions may be manufactured by processes well known in the art, for example, by means of conventional mixing, dissolving, granulating, drageemaking, levigating, emulsifying, encapsulating, entrapping or lyophilizing processes.

The pharmaceutical compositions may be formulated in conventional manner using one or more physiologically acceptable carriers comprising excipients and auxiliaries which facilitate processing of the active compounds into preparations which can be used pharmaceutically. Proper formulation is dependent upon the route of administration chosen.

For injection, the present compositions may be formulated in aqueous solutions, preferably in physiologically compatible buffers such as Hanks' solution, Ringer's solution, or physiological saline buffer. For transmucosal administration, penetrants appropriate to the barrier to be permeated are used in the formulation. Such penetrants are generally known in the art.

For oral administration, the present compositions may be formulated by combining the active compounds with pharmaceutically acceptable carriers well known in the art. Such carriers enable the compounds of the invention to be formulated as tablets, pills, lozenges, dragees, capsules, liquids, gels, syrups, slurries, suspensions and the like, for oral ingestion by a patient. Pharmaceutical preparations for oral use can be made using a solid excipient, optionally grinding the resulting mixture, and processing the mixture of granules, after adding other suitable auxiliaries if desired, to obtain tablets or dragee cores. Useful excipients are, in particular, fillers such as sugars, including lactose, sucrose, mannitol, or sorbitol, cellulose preparations such as, for example, maize starch, wheat starch, rice starch and potato starch and other materials such as gelatine, gum tragacanth, methyl cellulose, hydroxypropylmethyl-cellulose, sodium carboxymethylcellulose, and/or polyvinyl-pyrrolidone (PVP). If desired, disintegrating agents may be added, such as cross-linked polyvinyl pyrrolidone, agar, or alginic acid. A salt such as sodium alginate may also be used.

Dragee cores are provided with suitable coatings. For this purpose, concentrated sugar solutions may be used which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical compositions which can be used orally include push-fit capsules made of gelatine, as well as soft, sealed capsules made of gelatine and a plasticizer, such as glycerol or sorbitol. The push-fit capsules can contain the active ingredients in admixture with a filler such as lactose, a binder such as starch, and/or a lubricant such as talc or magnesium stearate and, optionally, stabilizers. In soft capsules, the active compounds may be dissolved or suspended in suitable liquids, such as fatty oils, liquid paraffin, or liquid polyethylene glycols. Stabilizers may be added in these formulations, also.

The compositions may also be formulated for parenteral administration, e. g., by bolus injection or continuous infusion. Formulations for injection may be presented in unit dosage form, e. g., in ampoules or in multi-dose containers, with an added preservative. The compositions may take such forms as suspensions, solutions or emulsions in oily or aqueous vehicles, and may contain formulating materials such as suspending, stabilizing and/or dispersing agents.

Pharmaceutical compositions for parenteral administration include aqueous solutions of a water soluble form, such as, without limitation, a salt, of the active compound.

Additionally, suspensions of the compositions may be prepared in a lipophilic vehicle. Suitable lipophilic vehicles include fatty oils such as sesame oil, synthetic fatty acid esters such as ethyl oleate and triglycerides, or materials such as liposomes. Aqueous injection suspensions may contain substances which increase the viscosity of the suspension, such as sodium carboxymethyl cellulose, sorbitol, or dextrane. Optionally, the suspension may also contain suitable stabilizers and/or agents that increase the solubility of the compounds to allow for the preparation of highly concentrated solutions.

Alternatively, the compositions may be in powder form for constitution with a suitable vehicle, e. g., sterile, pyrogen-free water, before use.

The compositions may also be formulated in rectal compositions such as suppositories or retention enemas, using, e.g., conventional suppository bases such as cocoa butter or other glycerides.

In addition to the formulations described previously, the compositions may also be formulated as depot preparations. Such long acting formulations may be administered by implantation (for example, subcutaneously or intramuscularly) or by intramuscular injection. The compositions may be formulated for this route of administration with suitable polymeric or hydrophobic materials (for instance, in an emulsion with a pharmacologically acceptable oil), with ion exchange resins, or as a sparingly soluble derivative such as, without limitation, a sparingly soluble salt.

A non-limiting example of a pharmaceutical carrier for the hydrophobic compounds of the invention is a cosolvent system comprising benzyl alcohol, a nonpolar surfactant, a water-miscible organic polymer and an aqueous phase such as the VPD co-solvent system. VPD is a solution of 3% w/v benzyl alcohol, 8% w/v of the nonpolar surfactant Polysorbate 80, and 65% w/v polyethylene glycol 300, made up to volume in absolute ethanol. The VPD co-solvent system (VPD: D5W) consists of VPD diluted 1:1 with a 5% dextrose in water solution. This cosolvent system dissolves hydrophobic compounds well, and itself produces low toxicity upon systemic administration.

Naturally, the proportions of such a co-solvent system may be varied considerably without destroying its solubility and toxicity characteristics. Furthermore, the identity of the co-solvent components may be varied: for example, other low toxicity nonpolar surfactants may be used instead of Polysorbate 80, the fraction size of polyethylene glycol may be varied, other biocompatible polymers may replace polyethylene glycol, e.g., polyvinyl pyrrolidone, and other sugars or polysaccharides may substitute for dextrose.

Alternatively, other delivery systems for hydrophobic pharmaceutical compounds may be employed. Liposomes and emulsions are well known examples of delivery vehicles or carriers for hydrophobic drugs. In addition, certain organic solvents such as dimethylsulfoxide also may be employed, although often at the cost of greater toxicity.

Additionally, the compounds may be delivered using a sustained-release system, such as semipermeable matrices of solid hydrophobic polymers containing the compositions.

Various sustained-release materials have been established and are well known by those skilled in the art. Sustained-release capsules may, depending on their chemical nature, release the compositions for a few weeks up to over 100 days. Depending on the chemical nature and the biological stability of the compositions, additional strategies for stabilization may be employed.

The pharmaceutical compositions herein also may comprise suitable solid or gel phase carriers or excipients. Examples of such carriers or excipients include, but are not limited to, calcium carbonate, calcium phosphate, various sugars, starch, cellulose derivatives, gelatine, and polymers such as polyethylene glycols.

Pharmaceutical compositions suitable for use in the present invention include compositions wherein the compositions are contained in a therapeutically effective amount sufficient to achieve the intended purpose, e. g., the treatment or prevention of a bacterial infection.

More specifically, a therapeutically effective amount means an amount of the composition effective to prevent, alleviate or ameliorate symptoms of bacterial infection or prolong the survival of the subject being treated.

Determination of a therapeutically effective amount is well within the capability of those skilled in the art, especially in light of the detailed disclosure provided herein. For example, the therapeutically effective amount or dose can be estimated initially from the described assays. Then, the dosage can be formulated for use in animal models so as to achieve a circulating concentration range that includes the MIC as determined in the experiments (i.e., the minimum concentration of the test compound which achieves inhibition of bacterial growth). Such information can then be used to more accurately determine useful doses in humans.

Toxicity and therapeutic efficacy of the compounds described herein can be determined by standard pharmaceutical procedures in cell cultures or experimental animals, e.g., by determining the MIC for a subject antibiotic. The data obtained from these cell culture assays and animal studies can be used in formulating a range of dosage for use in humans. The dosage may vary depending upon the dosage form employed and the route of administration utilized. The exact formulation, route of administration and dosage can be chosen by the individual physician in view of the patient's condition.

Dosage amount and interval may be adjusted individually to provide plasma levels of the active species which are sufficient to maintain the anti-bacterial effect. These plasma levels are referred to as minimal effective concentrations (MECs).

Dosages necessary to achieve the MEC will depend on individual characteristics and route of administration. HPLC assays or bioassays can be used to determine plasma concentrations. Dosage intervals can also be determined using MEC value. Compounds should be administered using a regimen that maintains plasma levels above the MEC for 10-90% of the time, preferably between 30-90% and most preferably between 50-90%. In cases of local administration or selective uptake, the effective local concentration of the drug may not be related to plasma concentration and other procedures known in the art may be employed to determine the correct dosage amount and interval.

The amount of a pharmaceutical composition administered may, of course, be dependent on the subject being treated, the severity of the affliction, the manner of administration, the judgment of the prescribing physician, etc.

The pharmaceutical compositions may, if desired, be presented in a pack or dispenser device, such as a kit approved by a regulatory authority, such as EMEA or FDA, which may contain one or more unit dosage forms containing the active composition. The pack may for example comprise metal or plastic foil, such as a blister pack. The pack or dispenser device may be accompanied by instructions for administration.

The pack or dispenser may also be accompanied by a notice associated with the container in a form prescribed by a governmental agency regulating the manufacture, use or sale of pharmaceuticals, which notice is reflective of approval by the agency of the form of the compositions or of human or veterinary administration.

Pharmaceutical compositions comprising the present compositions formulated in a compatible pharmaceutical carrier may also be prepared, placed in an appropriate container, and labeled for treatment of an indicated condition.

As used herein, a “physiologically/pharmaceutically acceptable carrier” refers to a carrier or diluent that does not cause significant irritation to an organism and does not abrogate the biological activity and properties of the administered compound.

A “pharmaceutically acceptable excipient” refers to an inert substance added to a pharmaceutical composition to further facilitate administration of a compound. Examples, without limitation, of excipients include calcium carbonate, calcium phosphate, various sugars and types of starch, cellulose derivatives, gelatine, vegetable oils and polyethylene glycols.

In a further aspect, the present compositions or pharmaceutical compositions may be used for the manufacture of a medicament for treating or preventing a bacterial infection in a subject or an organism. In this context, the bacterial infection may be caused by a Gram negative or a Gram positive bacterium. The bacterial infection may, for example, be caused by bacteria of the genus Acinetobacter, Actinomyces, Aeromonas, Bordetella, Borrelia, Brucella, Burkholderia, Campylobacter, Chlamydia, Clostridium, Corynebacterium, Enterococcus, Erwinia, Escherichia, Francisella, Haemophilus, Helicobacter, Klebsiella, Legionella, Leptospira, Listeria, Mycobacterium, Mycoplasma, Neisseria, Pseudomonas, Rickettsia, Salmonella, Shigella, Staphylococcus, Streptococccus, Treponema, Veillonella, Vibrio or Yersinia. In various embodiments, the infection is caused by Bacillus cereus, Bacillus thuringiensis, Neisseria subflava, Staphylococcus aureus, Mycobacterium smegmatic, or Pseudomonas aeruginosa. The subject affected by the bacterial infection may be a mammal, such as a human being.

In line with the above, the compositions or pharmaceutical compositions may, for example, be applied to the bacteria described above. In the following it is explained that the compositions or pharmaceutical compositions may be used as antibacterial agents in various applications.

For example, the compositions or pharmaceutical compositions are useful for the treatment of mammalian in particular human diseases caused by bacteria through interference of bacterial physiology. Such diseases include endocarditis, respiratory and pulmonary infections (preferably in immunocompromized and cystic fibrosis patients), bacteremia, central nervous system infections, ear infections including external otitis, eye infections, bone and joint infections, urinary tract infections, gastrointestinal infections and skin and soft tissue infections including wound infections, pyoderma and dermatitis. Furthermore, the compositions or pharmaceutical compositions can, for example, also be used for the treatment of pulmonary infections, gastroenteritis and wound infections, sepsis in tropical and subtropical areas, diarrhoea with blood and haemolytic uremic syndrome (HUS), yersiniosis, and transfusion-related sepsis and fistulous pyoderma, to name only a few.

In one embodiment, the present compositions or pharmaceutical compositions may be used for the manufacture of a medicament for treating or preventing a bacterial infection caused by Bacillus thuringiensis, wherein the antibiotic is kanamycin, gentamicin, ampicillin, or rifampicin, and wherein the fatty acid is DSF.

In another embodiment, the present compositions or pharmaceutical compositions may be used for the manufacture of a medicament for treating or preventing a bacterial infection caused by Neisseria subflava, wherein the antibiotic is kanamycin or gentamicin, and wherein the fatty acid is DSF.

In a further embodiment, the present compositions or pharmaceutical compositions may be used for the manufacture of a medicament for treating or preventing a bacterial infection caused by Staphylococcus aureus, wherein the antibiotic is kanamycin, gentamicin, or ampicillin, and wherein the fatty acid is DSF.

In yet further embodiment, the present compositions or pharmaceutical compositions may be used for the manufacture of a medicament for treating or preventing a bacterial infection caused by Mycobacterium smegmatic, wherein the antibiotic is kanamycin or gentamicin, and wherein the fatty acid is DSF.

Another aspect relates to a method of treating or preventing a bacterial infection in a subject, comprising administering a therapeutically effective amount of the present composition or the pharmaceutical composition to a subject in need thereof. The route of administration and the therapeutically effect amount are as defined above.

In yet another aspect, a method of supporting antibiotic therapy or prophylaxis in a subject, comprising administering a therapeutically effective amount of a fatty acid represented by formula (I), a stereoisomer, a salt or an ester thereof,

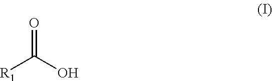

wherein R₁ is a substituted or unsubstituted aliphatic group, and
at least one antibiotic to a subject in need thereof, wherein the antibiotic is selected from the group consisting of an aminoglycoside antibiotic, a beta-lactam antibiotic, an ansamycin antibiotic, a macrolide antibiotic, a sulfonamide antibiotic, a quinolone antibiotic, an oxazolidinone antibiotic, a glycopeptide antibiotic, and a mixture thereof. The route of administration and the therapeutically effect amount are as defined above.

In various embodiments, the fatty acid is administered in combination with the at least one antibiotic. In other words, the fatty acid and the at least one antibiotic are administered simultaneously to the subject.

In alternative embodiments, the fatty acid and the at least one antibiotic are administered sequentially. In other words, the fatty acid and the at least one antibiotic are administered to the subject one after the other. The order of the administration is immaterial, meaning that the fatty acid may be administered prior to the at least one antibiotic, or vice versa.

By “comprising” it is meant including, but not limited to, whatever follows the word “comprising”. Thus, use of the term “comprising” indicates that the listed elements are required or mandatory, but that other elements are optional and may or may not be present.

By “consisting of” is meant including, and limited to, whatever follows the phrase “consisting of”. Thus, the phrase “consisting of” indicates that the listed elements are required or mandatory, and that no other elements may be present.

The inventions illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

By “about” in relation to a given numerical value, such as for temperature and period of time, it is meant to include numerical values within 10% of the specified value.

The invention has been described broadly and generically herein. Each of the narrower species and sub-generic groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

In order that the invention may be readily understood and put into practical effect, particular embodiments will now be described by way of the following non-limiting examples.

### Examples

In the examples described below, it is reported that in the presence of the diffusible signal factor (DSF) and its structural analogues (collectively as DSF-family molecules), which were identified in a range of bacterial species as quorum sensing signals (Barber et al., 1997; Wang et al., 2004; Boon et al., 2008; Deng et al., 2011), the minimum inhibitory concentration (MIC) of several antibiotics against B. cereus were significantly reduced. Furthermore, supplementation of DSF also enhances the antibiotic-susceptibility of B. thuringiensis, N. subflava, S. aureus, M. smegmatis and P. aeruginosa. These results have illustrated the promising potentials of DSF-family molecules as antibiotic adjuvants for the control of bacterial infections. The structure of DSF (cis 11-methyl-2-dodecenoic acid) is given in Table 1, the compound identified as “DSF”, along with its structural analogues. DSF is originally identified from the plant bacterial pathogen Xanthomonas campestris pv. Campestris (Xcc).

Materials and Methods

MIC Assay.

Minimum inhibitory concentration (MIC) was determined according to the 2003 guidelines of Clinical and Laboratory Standards Institute (CLSI) using 96-well microtiter plates. The two-fold dilution series of antibacterial agents or antibiotics were prepared with distilled water, methanol, or dimethyl sulfoxide (DMSO). Overnight cultures of bacteria were inoculated at an OD₆₀₀ of 0.025 in Luria-Bertani (LB) broth supplemented with the respective antibiotic in the absence or presence of DSF-family molecules (Table 1). One hundred microliters of inoculated culture were grown in each well at 37° C. with shaking at 150 rpm for 24 hours. MIC was defined as the lowest concentration of antibiotic in which bacterial growth in the well was not measureable by determination of the turbidity at 600 nm.

Bacterial Growth Analysis.

Overnight bacterial cultures grown in LB broth was inoculated in the same medium to an OD₆₀₀ of 0.025; the medium was supplemented with a respective antibiotic and DSF-family signals were added in a final concentration of 50 μM or otherwise indicated. Three hundred microliters of inoculated culture were grown in each well at 37° C. in a low intensity shaking model using the Bioscreen-C Automated Growth Curves Analysis System (OY Growth Curves AB Ltd, Finland).

Results and Discussion

DSF-Family Molecules Enhance the Antibiotic Susceptibility of B. cereus.

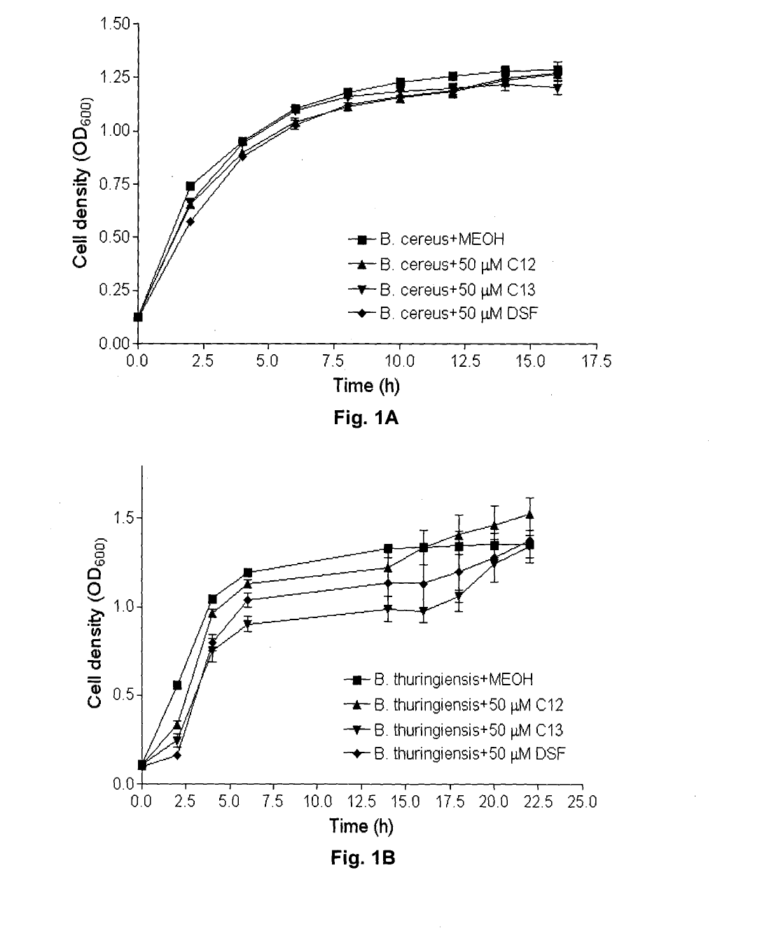

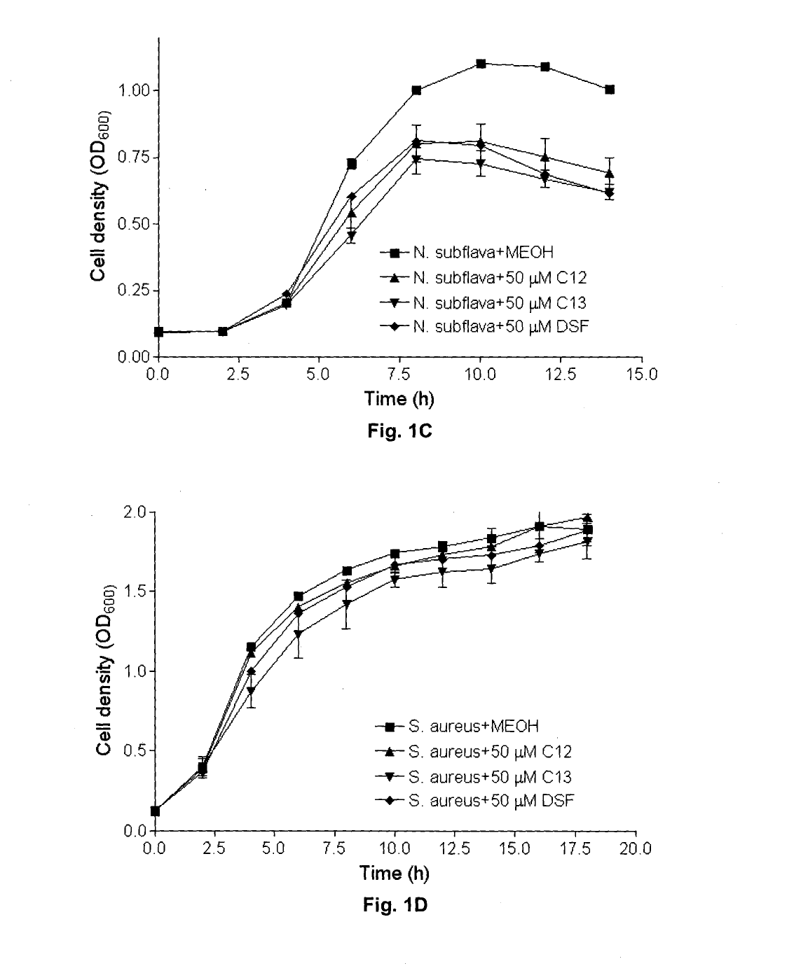

B. cereus is a common human pathogen and causes foodborne illness such as nausea, vomiting and diarrhea (Kotiranta et al., 2000). To test the effect of DSF-family molecules on the antibiotic susceptibility of B. cereus, DSF-family molecules were added to growth medium at a final concentration of 50 μM and the MICs of gentamicin, kanamycin, rifampicin and ampicillin against B. cereus were tested. Except for T8, T15 and C8, all the other DSF-family molecules showed a significantly synergistic effect with gentamicin (Table 2), which is an aminoglycoside antibiotic and inhibits bacterial protein synthesis mainly through binding with the 30S ribosomal subunit. For example, addition of T14 or C15 decreased the MIC of gentamicin against B. cereus from 8.0 μg/ml to 0.0625 μg/ml, which represented a 128-fold difference (Table 2). Similarly, combination of all the tested DSF-family molecules and kanamycin enhanced the antibiotic susceptibility of B. cereus to kanamycin from 2- to 64-fold with T14 showed the strongest synergistic activity (Table 3). Kanamycin is also an aminoglycoside antibiotic, which interacts with the 30S subunit of prokaryotic ribosomes and inhibits protein synthesis. Compared to the strong synergistic effect on gentamicin and kanamycin, DSF-family molecules showed a moderate effect on rifampicin, addition of DSF-family molecules increased the antibiotic sensitivity of B. cereus up to 4-fold (Table 4). Rifampicin inhibits the DNA-dependent RNA polymerase in bacterial cells, thus preventing gene transcription to RNA and subsequent translation to proteins. For ampicillin, which acts as a competitive inhibitor on transpeptide involved in bacterial membrane biosynthesis, only T11, T14, C11 and C15 increased the susceptibility of B. cereus to the antibiotic from 2- to 32-fold (Table 5). Among them C15 was the best drug adjuvant with ampicillin. Furthermore, the effect on growth curve of B. cereus by exogenous addition of DSF-family molecules was also tested. As shown in FIG. 1A, addition of C12, C13 and DSF at a final concentration of 50 μM did not affect the bacterial growth rate, demonstrating that these DSF molecules are non-toxic to B. cereus.

In general, the data showed that the unsaturated long chain DSF-family molecules have better synergistic activity with antibiotics, especially the aminoglycoside antibiotics, than the short chain and saturated molecules. These results indicate that DSF-family molecules effectively reduce the antibiotic resistance of B. cereus, and the synergistic effect is dependent on the structure of DSF-family molecules and may be related to the inhibitory mechanisms of antibiotics.

The Synergistic Effect of Dsf-Family Molecules with Antibiotics on B. cereus is Dosage-Dependent.

To determine whether the synergistic effect of DSF-family molecules with antibiotics is related to their dosages, C13 and DSF were supplemented to growth medium at different final concentrations and test the MICs of gentamicin and kanamycin against B. cereus. Results indicated that effect of C13 and DSF on the bacterial sensitivity to gentamicin and kanamycin in B. cereus was dependent on their final concentration (Tables 6, 7, 8, 9). C13 at a final concentration from 10 μM to 50 μM enhanced the antibiotic susceptibility of B. cereus to gentamicin by 2- to 32-fold, respectively (Table 6). Addition of DSF at a final concentration from 5 μM to 50 μM also had similar effect on the bacterial sensitivity to gentamicin (Table 7). We then continued to test the synergistic effect of different concentrations of C13 and DSF with kanamycin. As shown in Table 8 and Table 9, addition of C13 at the same concentration range enhanced the kanamycin susceptibility of B. cereus by 2- to 16-fold, and combination of different concentrations of DSF with kanamycin increased the synergy effect by 1.3- to 16-fold.

The Combination Effect of DSF with Antibiotics on Other Bacteria.

To study whether DSF-family molecules could also influence the antibiotic susceptibility in other bacterial pathogens, we tested the effect of DSF on the MICs of antibiotics against B. thuringiensis, N. subflava, S. aureus, and M. smegmatis. Among them, B. thuringiensis belongs to Bacillus cereus group and has been used as a bio-pesticide for many years (Addison, 1993; Ali, 1980, 1981). It is very closely related to the other two members of Bacillus cereus group, B. anthracis and B. cereus, which are important human pathogens to cause anthrax and a foodborne illness, respectively (Helgason et al., 2000). N. subflava is a rare opportunistic pathogen and has been associated with endocarditis, bacteremia, meningitis, septic arthritis, endophthalmitis, and septicemia (Pollack et al., 1984). S. aureus is the most common cause of staph infections. It can cause a range of serious illnesses such as pneumonia, meningitis, osteomyelitis, endocarditis, toxic shock syndrome (TSS), chest pain, bacteremia, and sepsis (Kluytmans et al., 1997). M. smegmatis is a useful model organism for research analysis of other Mycobacteria species. It is generally considered to be a non-pathogenic bacterium, however, in some very rare cases it may also cause diseases (Collins, 1989).

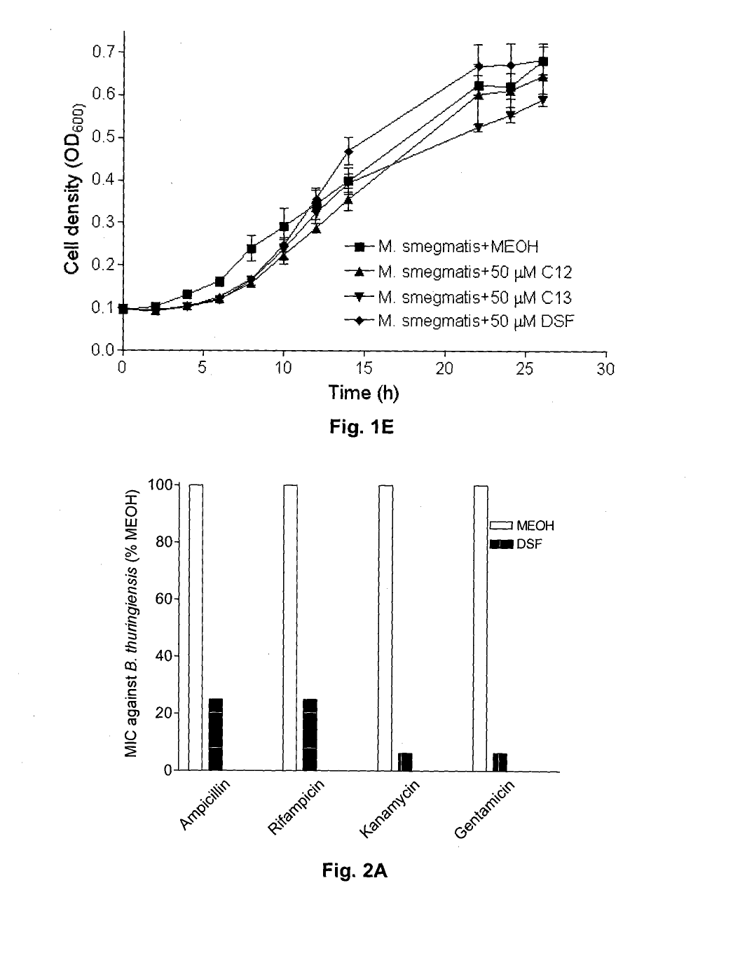

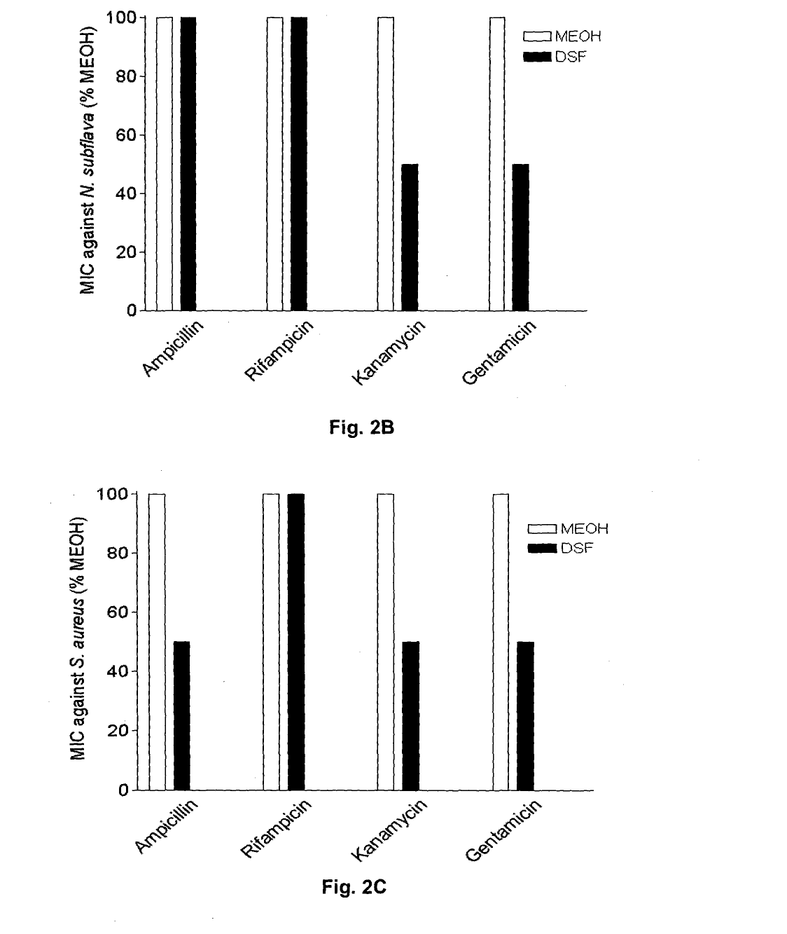

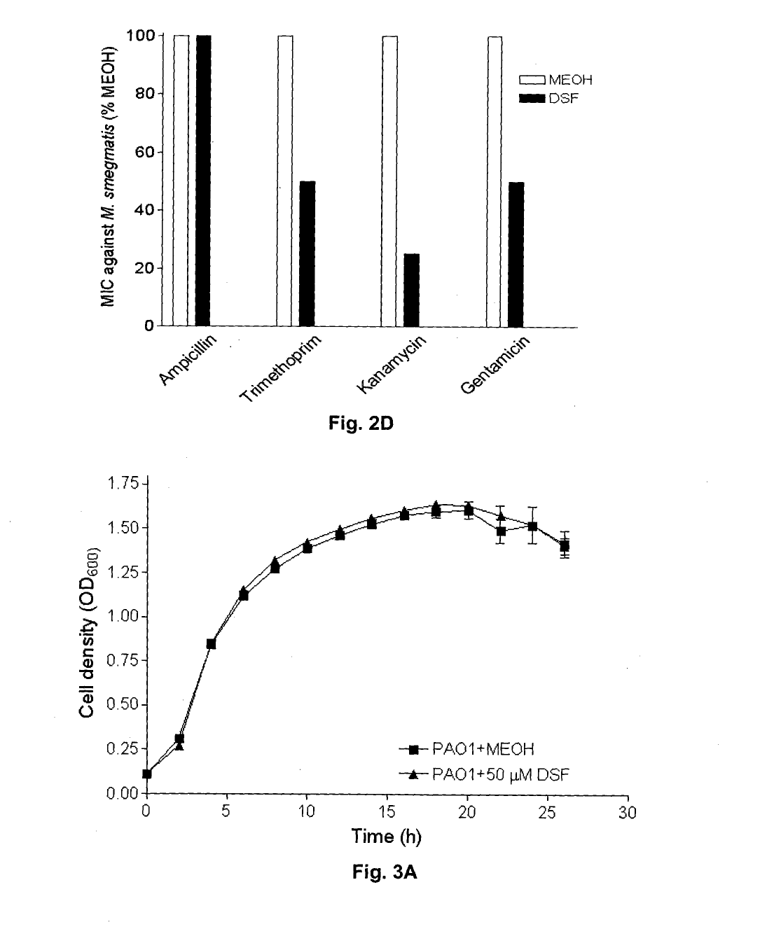

As shown in FIG. 2A, addition of DSF at a final concentration of 50 μM decreased the MICs of ampicillin, rifampicin, kanamycin and gentamicin against B. thuringiensis by 75%, 75%, 93.75% and 93.75%, respectively. Addition of DSF reduced the MICs of kanamycin and gentamicin against N. subflava by about 50%, but did not affect the MICs of ampicillin and rifampicin (FIG. 2B). We then continued to test the synergistic effect of DSF with antibiotics against S. aureus and M. smegmatis. Inclusion of DSF at a final concentration of 50 μM caused the reduction of the MICs of ampicillin, kanamycin and gentamicin by 50% (FIG. 2C) while for M. smegmatis, addition of DSF enhanced its susceptibility to trimethoprim, kanamycin and gentamicin by 50%, 25%, and 50%, respectively (FIG. 2D). These results indicate that the synergistic activity of DSF with antibiotics against bacteria is a conserved feature. However, different from the effect on B. cereus, exogenous addition of DSF-family molecules might slightly affect the growth rate of B. thuringiensis, S. aureus, and M. smegmatis (FIGS. 1B, 1D, 1E). The growth inhibitory effect of the DSF-family molecules became obvious when tested against N. subflava (FIG. 1C).

DSF Decreases the Antibiotic Tolerance of P. aeruginosa.

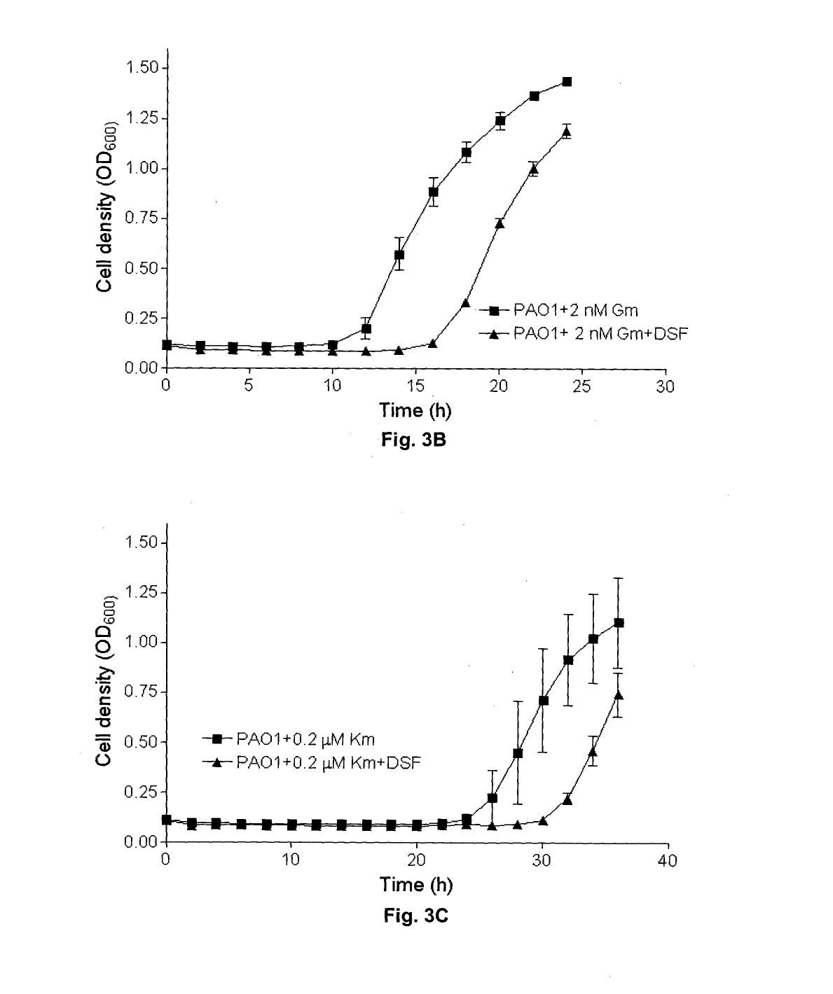

P. aeruginosa is a major source of opportunistic infections in both immunocompromised individuals and cystic fibrosis patients (Bodey et al., 1983; Richards et al., 2000). It appeared to be less responsive to DSF than other tested bacterial pathogen in our preliminary MIC assay. We therefore used the Bioscreen-C Automated Growth Curves Analysis System to determine the synergistic effect of DSF with antibiotics. Gentamicin and kanamycin were added to the culture of P. aeruginosa strain PAO1 at a final concentration of 2 nM and 0.2 μM, respectively, in the absence and presence of 50 μM DSF. Addition of 50 μM DSF did not affect the growth rate of PAO1 (FIG. 3A), but enhanced the susceptibility of P. aeruginosa to gentamicin and kanamycin (FIG. 3B, 3C). In the presence of gentamicin and kanamycin, the bacterial growth was initially inhibited but recovered at the 10^{th} and 22^{nd} hour post inoculation (FIG. 3B, 3C), whereas addition of DSF inhibited the recovery and delayed the bacterial growth by 6 and 8 hours, respectively (FIG. 3B, 3C).

In summary, it is showed that DSF-family molecules could significantly enhanced bacterial sensitivity to antibiotics, especially aminoglycoside antibiotics such as gentamycin and kanamycin. This synergistic effect is generic on both Gram-positive and Gram-negative bacteria with the Gram-positive bacteria being more sensitive to the activity of DSF-family molecules than the Gram-negative bacteria. The findings would be useful for reducing the dose of antibiotics, hence minimizing the side effects caused by antibiotics, and for slowing down the development of antibiotic resistance.

TABLE 1
Chemical Structures of DSF and its Derivatives used in the Examples

| Compound | Configuration | Structure |
|---|---|---|
| T8 | trans | 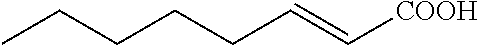 |
| T10 | trans | 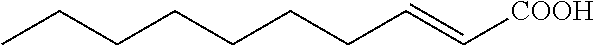 |
| T11 | trans | 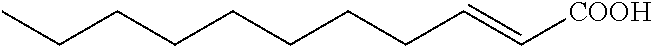 |
| T12 | trans | 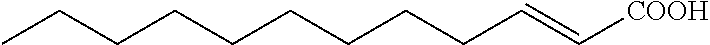 |
| T13 | trans | 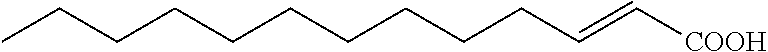 |
| T14 | trans | 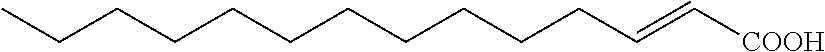 [SMILES: CCCCCCCCCCC/C=C/C(=O)O] |
| T15 | trans | 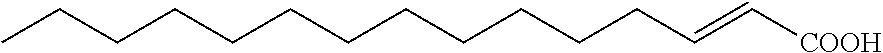 [SMILES: CCCCCCCCCCCC/C=C/C(=O)O] |
| C8 | cis | 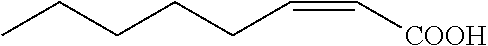 |
| C10 | cis | 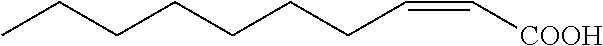 |
| C11 | cis | 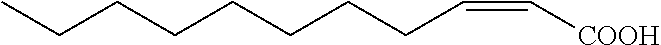 |
| C12 | cis | 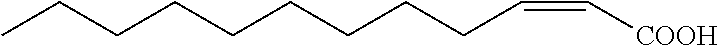 |
| DSF | cis | 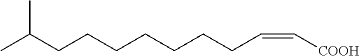 |
| C13 | cis | 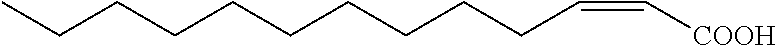 |
| C14 | cis | 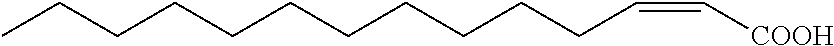 [SMILES: CCCCCCCCCCC/C=C\C(=O)O] |
| C15 | cis | 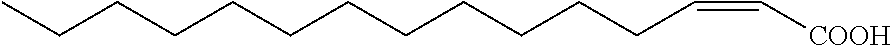 [SMILES: CCCCCCCCCCCC/C=C\C(=O)O] |
| S12 | NT | 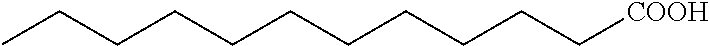 |

TABLE 2
Synergistic Effect of DSF-family Molecules on Gentamicin
against B. Cereus

MIC of Gentamicin
(μg/ml)

| DSF-family | | Standard | |
|---|---|---|---|
| molecules | Mean | Deviation | Fold change |
| MeOH | 8 | 0 | |
| T8 | 8 | 0 | 0 |
| T10 | 4 | 0 | 2 |
| T11 | 0.5 | 0 | 16 |
| T12 | 0.375 | 0.177 | 21.3 |
| T13 | 1 | 0 | 8 |
| T14 | 0.0625 | 0 | 128 |
| T15 | 8 | 0 | 0 |
| C8 | 8 | 0 | 0 |
| C10 | 4 | 0 | 2 |
| C11 | 0.5 | 0 | 16 |
| C12 | 1 | 0 | 8 |
| C13 | 0.25 | 0 | 32 |
| C14 | 0.25 | 0 | 32 |
| C15 | 0.0625 | 0 | 128 |
| DSF | 0.5 | 0 | 16 |
| S12 | 2 | 0 | 4 |

TABLE 3
Synergistic Effect of DSF-family Molecules on Kanamycin
against B. Cereus

MIC of Kanamycin
(μg/ml)

| DSF-family | | Standard | |
|---|---|---|---|
| molecules | Mean | Deviation | Fold Change |
| MeOH | 32 | 0 | |
| T8 | 16 | 0 | 2 |
| T10 | 8 | 0 | 4 |
| T11 | 4 | 0 | 8 |
| T12 | 2 | 0 | 16 |
| T13 | 6 | 2.828 | 5.3 |
| T14 | 0.5 | 0 | 64 |
| T15 | 16 | 0 | 2 |
| C8 | 16 | 0 | 2 |
| C10 | 8 | 0 | 4 |
| C11 | 2 | 0 | 16 |
| C12 | 8 | 0 | 4 |
| C13 | 2 | 0 | 16 |
| C14 | 1 | 0 | 32 |
| C15 | 1 | 0 | 32 |
| DSF | 2 | 0 | 16 |
| S12 | 4 | 0 | 8 |

TABLE 4
Synergistic Effect of DSF-family molecules on Rifampicin
against B. Cereus

MIC of Rifampicin
(μg/ml)

| DSF-family | | Standard | |
|---|---|---|---|
| molecules | Mean | Deviation | Fold Change |
| MeOH | 1 | 0 | |
| T8 | 1 | 0 | 0 |
| T10 | 0.5 | 0 | 2 |
| T11 | 0.375 | 0.177 | 2.6 |
| T12 | 0.75 | 0.353 | 1.3 |
| T13 | 0.5 | 0 | 2 |
| T14 | 0.75 | 0.353 | 1.3 |
| T15 | 0.5 | 0 | 2 |
| C8 | 0.5 | 0 | 2 |
| C10 | 0.375 | 0.177 | 2.6 |
| C11 | 0.25 | 0 | 4 |
| C12 | 0.375 | 0.177 | 2.6 |
| C13 | 0.375 | 0.177 | 2.6 |
| C14 | 0.5 | 0 | 2 |
| C15 | 0.25 | 0 | 4 |
| DSF | 0.5 | 0 | 2 |
| S12 | 0.5 | 0 | 2 |

TABLE 5
Synergistic Effect of DSF-family Molecules on Ampicillin
against B. Cereus

MIC of Ampicillin
(μg/ml)

| DSF-family | | Standard | |
|---|---|---|---|
| molecules | Mean | Deviation | Fold Change |
| MeOH | 2400 | 0 | |
| T8 | 2400 | 0 | 0 |
| T10 | 2400 | 0 | 0 |
| T11 | 300 | 0 | 8 |
| T12 | 2400 | 0 | 0 |
| T13 | 2400 | 0 | 0 |
| T14 | 1200 | 0 | 2 |
| T15 | 2400 | 0 | 0 |
| C8 | 2400 | 0 | 0 |
| C10 | 2400 | 0 | 0 |
| C11 | 150 | 0 | 16 |
| C12 | 2400 | 0 | 0 |
| C13 | 2400 | 0 | 0 |
| C14 | 2400 | 0 | 0 |
| C15 | 75 | 0 | 32 |
| DSF | 2400 | 0 | 0 |
| S12 | 2400 | 0 | 0 |

TABLE 6
Synergistic Effect of Different Concentrations of C13 on
Gentamicin against B. Cereus

MIC of Gentamicin
(μg/ml)

| Concentrations | | Standard | Fold |
|---|---|---|---|
| of C13 (μM) | Mean | Deviation | change |
| MeOH | 8 | 0 | |
| 5 | 8 | 0 | 0 |
| 10 | 4 | 0 | 2 |
| 20 | 2 | 0 | 4 |
| 30 | 1 | 0 | 8 |
| 40 | 0.375 | 0.177 | 21.3 |
| 50 | 0.25 | 0 | 32 |

TABLE 7
Synergistic Effect of Different Concentrations of
DSF on Gentamicin against B. Cereus

MIC of
Gentamicin (μg/ml)

| Concentrations | | Standard | Fold |
|---|---|---|---|
| of DSF (μM) | Mean | Deviation | change |
| MeOH | 8 | 0 | |
| 5 | 4 | 0 | 2 |
| 10 | 4 | 0 | 2 |
| 20 | 1.5 | 0.5 | 5.3 |
| 30 | 1 | 0 | 8 |
| 40 | 0.5 | 0 | 16 |
| 50 | 0.5 | 0 | 16 |

TABLE 8
Synergistic Effect of Different Concentrations of
C13 on Kanamycin against B. Cereus

MIC of
Kanamycin (μg/ml)

| Concentrations | | Standard | Fold |
|---|---|---|---|
| of C13 (μM) | Mean | Deviation | change |
| MeOH | 32 | 0 | |
| 5 | 16 | 0 | 2 |
| 10 | 16 | 0 | 2 |
| 20 | 4 | 0 | 8 |
| 30 | 4 | 0 | 8 |
| 40 | 2 | 0 | 16 |
| 50 | 2 | 0 | 16 |

TABLE 9
Synergistic Effect of Different Concentrations of
DSF on Kanamycin against B. Cereus

MIC of Kanamycin
(μg/ml)

| Concentrations | | Standard | Fold |
|---|---|---|---|
| of DSF (μM) | Mean | Deviation | change |
| MeOH | 32 | 0 | |
| 5 | 24 | 8 | 1.3 |
| 10 | 16 | 0 | 2 |
| 20 | 16 | 0 | 2 |
| 30 | 6 | 2 | 5.3 |
| 40 | 6 | 2 | 5.3 |
| 50 | 2 | 0 | 16 |

### REFERENCES AND NOTES

Aaron, S. D.; Ferris, W.; Henry, D. A.; Speert, D. P.; and Macdonald, N. E. Multiple combination bactericidal antibiotic testing for patients with cystic fibrosis infected with Burkholderia cepacia. Am. J. Respir. Crit. Care. Med. 2000, 161, 1206-1212.
Addison, J. A. Persistence and nontarget effects of Bacillus thuringiensis in soil: a review. Can. J. Forensic. Res. 1993, 23, 2329-2342.
Ali, A. Nuisance chironomids and their control: a review. Bull. Entomol. Soc. Am. 1980, 26, 3-16.
Ali, A. Bacillus thuringiensis serovar. israelensis (ABG-6108) against chironomids and some nontarget aquatic invertebrates. J. Invertebr. Pathol. 1981, 38, 264-272.
Athamna, A.; Athamna, M.; Nura, A.; Shlyakov, E.; Bast, D. J.; Farrell, D.; and Rubinstein, E. Is in vitro antibiotic combination more effective than single-drug therapy against anthrax? Antimicrob. Agents. Chemother. 2005, 49, 1323-1325.
Barber, C. E.; Tang, J. L.; Feng, J. X.; Pan, M. Q.; Wilson, T. J.; Slater, H.; Dow, J. M.; Williams, P.; Daniels, M. J. A novel regulatory system required for pathogenicity of Xanthomonas campestris is mediated by a small diffusible signal molecule. Mol. Microbiol. 1997, 24, 555-566.
Bodey, G. P.; Bolivar, R.; Fainstein, V.; Jadeja, L. Infections caused by Pseudomonas aeruginosa. Rev. Infect. Dis. 1983, 5, 279-313.
Boon, C.; Deng, Y.; Wang, L. H.; He, Y.; Xu, J. L.; Fan, Y.; Pan, S. Q.; Zhang, L. H. A novel DSF-like signal from Burkholderia cenocepacia interferes with Candida albicans morphological transition. ISME J. 2008, 2, 27-36.
Collins, F. M. Mycobacterial disease, immunosuppression, and acquired immunodeficiency syndrome. Clin. Microbiol. Rev. 1989, 2, 360-377.
Costanzo, M.; Baryshnikova, A.; Bellay, J. et al. The genetic landscape of a cell. Science. 2010, 327, 425-431.
Costerton, J. W.; Lewandowski, Z.; Caldwell, D. E.; Korber, D. R.; Lappin-Scott, H. M. Microbial biofilms. Annu. Rev. Microbiol. 1995, 49, 711-45.
Deng, Y.; Wu, J. E.; Tao, F.; and Zhang, L. H. Listening to a new language: DSF-based quorum sensing in Gram-negative bacteria. Chem. Rev. 2011, 111, 160-173.
Ejim, L.; AFarha, M.; Falconer, S. B.; Wildenhain, J.; Coombes, B. K.; Tyers, M.; Brown, E. D.; and Wright, G. D. Combinations of antibiotics and nonantibiotic drugs enhance antimicrobial efficacy. Nat. Chem. Biol. 2011, 24 Apr.
Giannini, A. J.; Black, H. R. Psychiatric, Psychogenic and Somatopsychic Disorders Handbook. Garden City, N.Y. Medical Examination Publishing Co., 1978. pp. 136-137.
Helgason, E.; Okstad, Q. A.; Caugant, D. A.; Johansen, H. A.; Fouet, A.; Mock, M.; Hegna, I.; Kolsto. Bacillus anthracis, Bacillus cereus, and Bacillus thuringiensis—one species on the basis of genetic evidence. Appl. Environ. Microbiol. 2000, 66, 2627-2630.
Kluytmans, J.; Belkum, A. V.; Verbrugh, H. Nasal carriage of Staphylococcus aureus: epidemiology, underlying mechanisms, and associated risks. Clin. Microbiol. Rev. 1997, 10, 505-20.
Kotiranta, A.; Lounatmaa, K; Haapasalo, M. Epidemiology and pathogenesis of Bacillus cereus infections. Microbes. Infect. 2000, 2, 189-98.
Kristiansen, J. E.; Hendricks, O.; Delvin, T.; Butterworth, T. S.; Aagaard, L.; Christensen, J. B.; Flores, V. C.; Keyzer, H. Reversal of resistance in microorganisms by help of non-antibiotics. J. Antimicrob. Chemother. 2007, 59, 1271-1279.
Lehtinen, J.; Lilius, E. M. Promethazine renders Escherichia coli susceptible to penicillin G: real-time measurement of bacterial susceptibility by fluoro-luminometry. Int. J. Antimicrob. Agents. 2007, 30, 44-51.
Le, T., and Bayer, A. S. Combination antibiotic therapy for infective endocarditis. Clin. Infect. Dis. 2003, 36, 615-621.
Livermore, D. M. The need for new antibiotics. Clinical. Microbiol. Infect. 2004, 10, 1-9.
Mazumdar, K.; Dastidar, S. G.; Park, J. H.; Dutta, N. K. The anti-inflammatory non-antibiotic helper compound diclofenac: an antibacterial drug target. Eur. J. Clin. Microbiol. Infect. Dis. 2009, 28, 881-891.
Parsons, A. B., et al. Integration of chemical-genetic and genetic interaction data links bioactive compounds to cellular target pathways. Nat. Biotechnol. 2004, 22, 62-69
Pfaller, M. A.; Jones, R. N.; Doerm, G. V.; Kugler, K. Bacterial pathogens isolated from patients with bloodstream infection: frequencies of occurrence and antimicrobial susceptibility patterns from the SENTRY antimicrobial surveillance program (United States and Canada, 1997). Antimicrob. Agents. Chemother. 1998, 42, 1762-1770.
Pollack, S.; Mogtader, A.; Lange, M. Neisseria subflava endocarditis. Case report and review of the literature. Am. J. Med. 1984, 76, 752-758.
Richards, M. J.; Edwards, J. R.; Culver, D. H.; Gaynes, R. P. Nosocomial infections in combined medical-surgical intensive care units in the United States. Infect: Control. Hosp. Epidemiol. 2000, 21, 510-515.
Slama, T. G.; Amin, A.; Brunton, S. A.; et al. A clinician's guide to the appropriate and accurate use of antibiotics: the Council for Appropriate and Rational Antibiotic Therapy (CARAT) criteria. Am. J. Med. 2005, 118 suppl, 1-6.
Sundin, D. P.; Sandoval, R.; Molitoris, B. A. Gentamicin Inhibits Renal Protein and Phospholipid Metabolism in Rats: Implications Involving Intracellular Trafficking. J. Am. Soc. Nephrol. 2001, 12, 114-123.
Tao, F.; Swarup, S.; Zhang, L. H. Quorum sensing modulation of a putative glycosyltransferase gene cluster essential for Xanthomonas campestris biofilm formation. Environ Microbiol. 2010, 12, 3150-70
Wang, L. H.; He, Y.; Gao, Y.; Wu, J. E.; Dong, Y. H.; He, C.; Wang, S. X.; Weng, L. X.; Xu, J. L.; Tay, L.; Fang, R. X.; Zhang, L. H. A bacterial cell-cell communication signal with cross-kingdom structural analogues. Mol. Microbiol. 2004, 51, 903-912.

1. A composition comprising:
an antibiotic selected from the group consisting of an aminoglycoside antibiotic, a beta-lactam antibiotic, an ansamycin antibiotic, a macrolide antibiotic, a sulfonamide antibiotic, a quinolone antibiotic, an oxazolidinone antibiotic, a glycopeptide antibiotic, and a mixture thereof; and
a fatty acid represented by formula (I), a stereoisomer, a salt or an ester thereof, 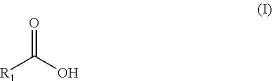
wherein R₁ is a substituted or unsubstituted aliphatic group.

2-3. (canceled)

4. The composition of claim 1, wherein the aliphatic group is unsaturated and R₁ is an alkenyl group, preferably R₁ is a C₅-C₂₀ alkenyl group.

5. (canceled)

6. The composition of claim 4, wherein the C₅-C₂₀ alkenyl group comprises at least one double bond.

7. The composition of claim 6, wherein the C₅-C₂₀ alkenyl group comprises at least one double bond at the carbon-2 position of the fatty acid.

8. (canceled)

9. The composition of claim 1, wherein the aliphatic group is saturated and R₁ is an alkyl group, preferably R₁ is a C₅-C₂₀ alkyl group.

10-11. (canceled)

12. The composition of claim 1, wherein the fatty acid is selected from the group consisting of:

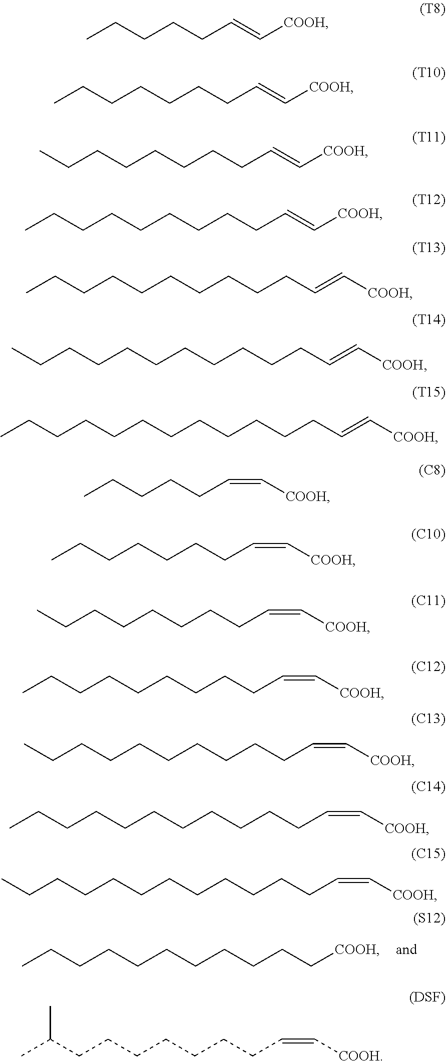

13. The composition of claim 1, wherein the antibiotic is an aminoglycoside antibiotic, preferably the amino glycoside antibiotic is kanamycin or gentamicin.

14. (canceled)

15. The composition of claim 13, wherein the aminoglycoside antibiotic is gentamicin and the fatty acid is T14 or C15.

16. The composition of claim 13, wherein the aminoglycoside antibiotic is kanamycin and the fatty acid is T14.

17. The composition of claim 1, wherein the antibiotic is a beta-lactam antibiotic, preferably the beta-lactam antibiotic is ampicillin.

18. (canceled)

19. The composition of claim 17, wherein the fatty acid is C15.

20. The composition of claim 1, wherein the antibiotic is an ansamycin antibiotic, preferably the ansamycin antibiotic is rifampicin.

21. (canceled)

22. The composition of claim 20, wherein the fatty acid is C11 or C15.

23-26. (canceled)

27. Use of a composition comprising
an antibiotic selected from the group consisting of an aminoglycoside antibiotic, a beta-lactam antibiotic, an ansamycin antibiotic, a macrolide antibiotic, a sulfonamide antibiotic, a quinolone antibiotic, an oxazolidinone antibiotic, a glycopeptide antibiotic, and a mixture thereof; and
a fatty acid represented by formula (I), a stereoisomer, a salt or an ester thereof, 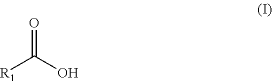
wherein R₁ is a substituted or unsubstituted aliphatic group, for the manufacture of a medicament for treating or preventing a bacterial infection.

28. (canceled)

29. The use of claim 27, wherein the bacterial infection is caused by Bacillus cereus, Bacillus thuringiensis, Neisseria subflava, Staphylococcus aureus, Mycobacterium smegmatic, or Pseudomonas aeruginosa.

30. The use of claim 29, wherein the bacterial infection is caused by Bacillus thuringiensis, wherein the antibiotic is kanamycin, gentamicin, ampicillin, or rifampicin, and wherein the fatty acid is DSF.

31. The use of claim 29, wherein the bacterial infection is caused by Neisseria subflava, wherein the antibiotic is kanamycin or gentamicin, and wherein the fatty acid is DSF.

32. The use of claim 29, wherein the bacterial infection is caused by Staphylococcus aureus, wherein the antibiotic is kanamycin, gentamicin, or ampicillin, and wherein the fatty acid is DSF.

33. The use of claim 29, wherein the bacterial infection is caused by Mycobacterium smegmatic, wherein the antibiotic is kanamycin or gentamicin, and wherein the fatty acid is DSF.

34. A method of treating or preventing a bacterial infection in a subject, comprising administering a therapeutically effective amount of a composition comprising
an antibiotic selected from the group consisting of an aminoglycoside antibiotic, a beta-lactam antibiotic, an ansamycin antibiotic, a macrolide antibiotic, a sulfonamide antibiotic, a quinolone antibiotic, an oxazolidinone antibiotic, a glycopeptide antibiotic, and a mixture thereof; and
a fatty acid represented by formula (I), a stereoisomer, a salt or an ester thereof, 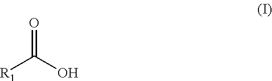
wherein R₁ is a substituted or unsubstituted aliphatic group, to a subject in need thereof.

35-37. (canceled)
